(19) United States
(12) Patent Application Publication
(10) Pub. No.: US 2016/0317395 A1
(43) Pub. Date: Nov. 3, 2016

(54) METAL BASED DENTAL ARTICLE HAVING A COATING

(71) Applicant: 3M INNOVATIVE PROPERTIES COMPANY, St. Paul, MN (US)

(72) Inventors: Bhaskar V. Velamakanni, Woodbury, MN (US); Steven Yu, St. Paul, MN (US)

(21) Appl. No.: 15/102,472

(22) PCT Filed: Dec. 8, 2014
(86) PCT No.: PCT/US2014/069104

§ 371 Date: Jun. 7, 2016

Related U.S. Application Data
(60) Provisional application No. 61/915,608, filed on Dec. 13, 2013.

(51) Int. Cl. A61K 6/04; A61C 5/10; A61C 13/00; A61C 7/20; A61C 8/00; A61C 13/107; A61K 6/00; A61C 7/14

(52) U.S. Cl. CPC A61K 6/04; A61K 6/0002; A61K 6/0017; A61C 5/10; A61C 7/14; A61C 7/20; A61C 8/0048; A61C 8/0013; A61C 8/008; A61C 13/0001; A61C 13/0003

(57) ABSTRACT

The present disclosure provides a metal based dental article and a method of forming the metal based dental article. The metal based dental article includes a base layer, a first coating on the base layer and a second coating on the first coating. The base layer is formed from a metal substrate selected from the group consisting of a stainless steel, a titanium alloy or a nickel titanium alloy. The first coating is formed from a metal selected from the group consisting of nickel, a nickel alloy, copper, a copper alloy, zinc, a zinc alloy, tin, a tin alloy, gold or a gold alloy, where one of the first coating or the surface of the base layer has a surface roughness (Rₐ) from 0.1 to 1.0 microns as measured according to ASTM B46.1-2009. The second coating is formed from a metal selected from the group consisting of rhodium, a rhodium alloy, ruthenium, a ruthenium alloy, cobalt, a cobalt alloy, palladium, or a palladium alloy. The second coating has a predetermined thickness that provides the metal based dental article with a matte finish and a whiteness, L*, of at least 80 of the CIELAB color system (L*, a*, B*). t,?

### FIELD OF DISCLOSURE

The disclosure relates to dental articles and in particular to metal based dental articles having a coating.

### BACKGROUND

Metal based dental articles are useful for treating of a variety of dental maladies. Such metal based dental articles have proven reasonably durable for both short-term and long-term dental treatments. Metal based dental articles are typically malleable, thereby allowing them to be modified chair side to adapt to the tooth structures of a particular patient and to ensure a secure installation. This degree of post-manufacturing freedom has made metal based dental articles such as crowns and orthodontic bands especially popular. Metal based dental crowns, particularly those made of stainless steel, are well suited for children, as their reasonable life span coincides with the natural exfoliation of children's teeth.

The metallic sheen of stainless steel, however, is not exactly esthetically appealing. Accordingly, attempts have been made to apply aesthetic coatings to stainless steel dental crowns, but these efforts have experienced little success in maintaining flexibility and durability. For example, a number of approaches have been taken to develop esthetic coatings on metal based dental articles with limited market success and/or penetration. Of those, NuSmile® crowns and Kinder Krowns® are notable for their veneered esthetic composite coatings on primary stainless steel crowns. Because these coatings tend to be bulky (>1000 μm), they require extensive tooth preparation for seating the metal based crown. So, hard tissue may have to be removed not only from the infected tooth, but also from the healthy adjacent teeth in order to accommodate these metal based crowns.

Therefore, there is a need in the art for metal based dental articles that have a coating that provides a more inconspicuous and aesthetic appearance in situ and provides a robust adhesion that not only has good wear resistance, but also provides flexibility for unhindered customization of the metal based dental article.

### SUMMARY

The present disclosure provides a metal based dental article having a coating that provides a more inconspicuous and aesthetic appearance in situ and provides a robust adhesion that not only has good wear resistance, but also provides flexibility for unhindered customization of the metal based dental article.

The metal based dental article of the present disclosure includes a base layer formed from a metal substrate selected from the group consisting of a stainless steel, a titanium alloy or a nickel titanium alloy, the base layer having a surface in the shape of the metal based dental article; a first coating on the surface of the base layer, the first coating formed from a metal selected from the group consisting of nickel, a nickel alloy, copper, a copper alloy, zinc, a zinc alloy, tin, a tin alloy, gold or a gold alloy, where one of the first coating or the surface of the base layer has a surface roughness (Rₐ) from 0.1 to 1.0 microns as measured according to ASTM B46.1-2009; and a second coating on the first coating, the second coating formed from a metal selected from the group consisting of rhodium, a rhodium alloy, ruthenium, a ruthenium alloy, cobalt, a cobalt alloy, palladium, or a palladium alloy, where the second coating has a predetermined thickness that provides the metal based dental article with a matte finish and a whiteness, L*, of at least 80 of the CIELAB color system (L*, a*, B*).

In one embodiment, the first coating can be formed from nickel and can have a satin finish. Alternatively, first coating can be formed from cobalt or a cobalt alloy, where the first coating has a satin finish. In an additional embodiment, the first coating can be formed from an alloy that includes copper, zinc and tin. The first coating can have a thickness from greater than zero microns to 25 microns. Preferably, the first coating has a thickness from 5 microns to 10 microns.

For the given embodiments, the second coating can be formed from rhodium. Alternatively, the second coating can be formed from rhodium and ruthenium. In an additional embodiment, the second coating can be formed from rhodium and palladium. The predetermined thickness of the second coating can be from greater than zero microns to 1 micron. Preferably, the predetermined thickness of the second coating is from 0.3 microns to 0.5 microns.

The present disclosure also provides for a method of forming the metal based dental article. The method of forming the metal based dental article includes providing a base layer formed from a metal substrate selected from the group consisting of a stainless steel, a titanium alloy or a nickel titanium alloy, the base layer having a surface in the shape of the metal based dental article; electroplating a first coating on the surface of the base layer using a metal selected from the group consisting of nickel, a nickel alloy, copper, a copper alloy, zinc, a zinc alloy, tin, a tin alloy, gold or a gold alloy; providing the first coating with a surface roughness (Rₐ) from 0.1 to 1.0 microns as measured according to ASTM B46.1-2009; and electroplating a second coating to a predetermined thickness on the first coating using a metal selected from the group consisting of rhodium, a rhodium alloy, ruthenium, a ruthenium alloy, cobalt, a cobalt alloy, palladium, or a palladium alloy, where the second coating provides the metal based dental article with a matte finish and a whiteness, L*, of at least 80 of the CIELAB color system (L*, a*, B*).

Providing the first coating with the surface roughness can include using a mechanical technique such as sandblasting the surface of the base layer to provide the surface roughness from 0.1 to 1.0 microns as measured according to ASTM B46.1-2009. As discussed herein, providing the first coating with the surface roughness can also include using chemical techniques (e.g., acid etching or selection of plating electrolyte) to provide the surface roughness from 0.1 to 1.0 microns as measured according to ASTM B46.1-2009.

In one embodiment, providing the base layer can include selecting stainless steel as the metal substrate; electroplating the first coating using nickel to form a satin nickel finish for the first coating; and electroplating the second coating using rhodium to form the matte finish and a whiteness, L*, of at least 80 of the CIELAB color system (L*, a*, B*). Electroplating the first coating can be to a thickness of greater than zero microns to 25 microns; and electroplating the second coating can be to a thickness of greater than zero microns to 1 micron.

The metal based dental article of the present disclosure can be implanted in the mouth of a patient. To this end, the present disclosure includes a kit having the metal based dental article of the present disclosure and at least one dental component selected from the group of a cement, an adhesive, an abrasive, an instrument, instructions for application of the metal dental article or a combination thereof.

The above summary of the present disclosure is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a bar graph showing the surface roughness (Rₐ) of Examples and Comparative Examples before and after the Toothbrush Wear Test.

### DEFINITIONS

As used herein, “a,” “an,” “the,” “at least one,” and “one or more” are used interchangeably. The term “and/or” means one, one or more, or all of the listed items. The recitations of numerical ranges by endpoints include all numbers subsumed within that range (e.g., 1 to 5 includes 1, 1.5, 2, 2.75, 3, 3.80, 4, 5, etc.).

As recited herein, all numbers can be considered to be modified by the term “about.”

As used herein a “metal based dental article” includes an article that can be adhered to (e.g., bonded), implanted in or attached to an oral surface (e.g., a tooth structure or a bone structure). Examples include, but are not limited to, dental restorations, restoratives, replacements, inlays, onlays, veneers, full crowns, partial crowns, bridges, bridge frameworks, dentures (e.g., partial or full dentures), implants, implant abutments, copings, implant healing caps, posts, temporary restorations, orthodontic appliances and devices including, but not limited to archwires, buccal tubes, brackets and bands and the like.

As used herein “whiteness” “L*” is defined by the “CIE 1976 (L*, a*, b*) color space” generally referred to as CIELAB color system (L*, a*, b*).

The term “comprising” and variations thereof (e.g., comprises, includes, etc.) do not have a limiting meaning where these terms appear in the description and claims.

As used herein, “continuous” means extending substantially across a target surface and including no deliberate gaps or interruptions other those inherent in the material.

### DETAILED DESCRIPTION

The present disclosure provides a metal based dental article having a coating that demonstrates robust adhesion, flexibility and a whiteness comparable to an average natural tooth whiteness. In addition, the thinness of the coating allows the metal based dental article of the present disclosure to provide the same ease of use and harmony with patient's occlusion and gingival health as uncoated metal based dental article. As a result, the metal based dental articles of the present disclosure may help to, among other things, provide a dental restoration (e.g., a crown) that can be used with minimal loss of hard tissue (e.g., infected tooth and adjacent healthy hard tissue).

The coating on the metal based dental article also demonstrates a resistance to wear that should provide the metal based dental article with desirable clinical performance. In addition, the coating on the metal based dental article according to the present disclosure can maintain desired flexibility such that it may be cut, bent, crimped, or otherwise manipulated by a practitioner without delamination or other failure of the coatings discussed herein. Therefore, use of the metal based dental article of the present disclosure may allow a dental or orthodontic practitioner to precisely modify the fit or shape of the metal based dental article without sacrificing performance or appearance.

The metal based dental article of the present disclosure includes a base layer formed from a metal substrate and a coating which includes a first coating on the surface of the base layer and a second coating on the first coating. The base layer has a surface in the shape of the metal based dental article. For example, the surface of the metal based dental article can have the shape of a full crown, a partial crown, a pediatric crown, a bridge, a denture, an implant, an implant abutment, an implant healing cap, a post, a temporary restoration, an orthodontic bracket, an arch wire or a part of any one of those articles. Other examples of metal based dental articles are provided herein.

The first coating and the second coating can provide the metal based dental article with a variety of finishes and whiteness (L*) values. As used herein a finish refers to the appearance of the metal based dental article that results from the method of forming the metal based dental article and its coatings. Examples of such finishes for the metal based dental article include a matte finish or a satin finish. A matte finish is one that is dull in appearance, where as a satin finish is a finish that is typically formed by scratch-brushing a polished metal surface to produce a soft sheen. In either case, the matte finish or the satin finish can be achieved by first imparting to one of or both of the base layer of the metal substrate and/or the first coating of the metal based dental article a surface roughness (Rₐ) from 0.1 to 1.0 microns as measured according to ASTM B46.1-2009. Methods for producing the surface roughness include either chemical or mechanical techniques, which will be discussed more fully herein.

The base layer is formed from a metal substrate selected from the group consisting of a stainless steel, a titanium alloy or a nickel titanium alloy. Examples of suitable stainless steel include, but are not limited to, American Iron and Steel Institute (AISI)-Types 304L stainless steel, or a 316 medical grade stainless steel (based on the AISI Classification of Chromium-Nickel Stainless Steels). Examples of suitable titanium alloys include, but are not limited to, Ti-6Al-4V, Ti-6Al-4V Extra Low Interstitial and Ti—Al—Nb grades of titanium alloy, as recognized by ASTM International. Examples of suitable nickel titanium alloys for biomedical devices and implants include, but are not limited to, those alloys having approximately equal atomic percentages of nickel and titanium (ASTM F2063-05).

The first coating is formed from a metal selected from the group consisting of nickel, a nickel alloy, copper, a copper alloy, zinc, a zinc alloy, tin, a tin alloy, gold or a gold alloy. For example, the first coating can be formed from electroless or electroplated nickel. As discussed and demonstrated herein, the first coating can be formed from nickel having a satin finish. In an alternative embodiment, the first coating can be formed from cobalt or a cobalt alloy, where this first coating can have a satin finish. The first coating can also be formed from an alloy that includes copper, zinc and tin.

Any suitable source of metal ions can be used to form the first coating. The metal ions in the bath can be derived from soluble metal salts that dissociate from each other in the bath solution. Non-limiting examples of suitable metal salts for nickel ions include nickel sulfate, nickel chloride, and nickel sulfamate. Non-limiting examples of suitable metal salts for copper ions include cupric and cuprous salts such as cuprous chloride or sulfate. Non-limiting examples of suitable metal salts for zinc ions can include acid, alkaline, and cyanide, among others. Other suitable salts useful for depositing other metals are known in the deposition art. Different types of metal salts can be used if a metal alloy (e.g., a nickel alloy) is to be formed.

Alternatively, commercially available compositions from which the first coating can be formed include those sold under the trade designator CL-Satin Nickel (Uyemura Intl Co., Southington, Conn.). Examples of compositions from which a copper, tin and zinc alloy can be formed include alkaline-cyanide electroplating baths containing 53 weight percent (wt. %) copper, 15 wt. % tin, and 32 wt. % zinc, based on the total weight of the metal in the composition. Other commercially available examples of the metal for the first coating are sold under the trade designators Miralloy 2844 (Uyemura Intl Co.), TechniWhite (Prodigy Surface Tech., Santa Clara, Calif.), among others.

The baths in which the first coating takes place can include at least the metal ions, provided herein, and plating additives. The solvent that is used in these baths can include water so that the bath is aqueous. Generally, deposition conditions such as the pH, deposition time, bath constituents, and deposition temperature can be chosen by those of ordinary skill in the art in order to achieve the desired thickness and composition of the first coating. For the various embodiments, the deposition conditions can be selected to provide the first coating with a thickness from greater than zero microns to 25 microns. Preferably, the first coating has a thickness from 5 microns to 10 microns. For the various embodiments, the first coating can be continuous over the entire surface (e.g., outer surface) of the base layer.

If desired, a strike layer can be deposited on the surface of the base layer prior to depositing the first coating. Examples of such strike layers include those suitable for use with chromium-nickel stainless steel, such as AURUNA® 311 (Umicore Galvanotechnik GmbH, Germany) for electroplating rhodium-ruthenium on either stainless steel or Ni—Ti alloy) and Woods Nickel Strike (e.g., 119-475 g/L of nickel chloride (Uyemura Intl Co.) for CL-Satin Ni deposition. Appropriate submersion times in these strike baths can be approximately 1 to 4 minutes with an application of approximately 20 to 200 amps per square foot (ASF) current density for uniform thickness of 1 to 4 micron.

The second coating is formed from a metal selected from the group consisting of rhodium, a rhodium alloy, ruthenium, a ruthenium alloy, cobalt, a cobalt alloy, palladium or a palladium alloy. Of these metals, cobalt and cobalt alloy can be chosen for a variety of reasons, including economic, aesthetic, physical and/or chemical reasons. The second coating has a predetermined thickness that provides the metal based dental article with a whiteness, L*, of at least 80 of the CIELAB color system (L*, a*, B*) without affecting the matte finish of the first coating.

In addition to imparting whiteness, the choice of the metal used in the second layer can provide added protection to the coated layers in terms of hardness, density, chemical resistance and mechanical properties, among other properties. For example, high density metal or metal alloys with sufficient hardness and modulus can be effective as a wear-resistant coating. In addition, in combination with their Noble character, the coated layer is not only hypoallergenic but also may prevent diffusion of metal ions such as Ni from the base metal structure or submerged coatings (e.g., the first coating and the second coating on the surface of the base layer may help to arrest any potential leaching of allergic metal ions from the base layer and first coating such as Ni).

Preferably, the second coating is formed from rhodium. An additional preferable embodiment is where the second coating is formed from rhodium (Rh) and ruthenium (Ru). Another preferable embodiment is where the second coating is formed from Rh and palladium. Examples of suitable molar ratios of Rh and Ru for forming the second coating include a 4:1 to a 1:1 molar ratio (Rh:Pd) for forming the second coating.

Any suitable source of metal ions can be used forming the second coating. The metal ions in the bath can be derived from soluble metal salts that dissociate from each other in the bath solution. Non-limiting examples of suitable metal salts for Rh ions include sulfate and phosphate. Non-limiting examples of suitable metal salts for Ru ions include chloride and sulfate. Non-limiting examples of suitable metal salts for cobalt ions can include sulfate and chloride. Other suitable salts useful for depositing other metals are known in the deposition art. Different types of metal salts can be used if a metal alloy (e.g., a Rh alloy) is to be formed.

Alternatively, commercially available compositions from which the second coating can be formed include those sold under the trade designator Rhoduna Alloy-1 (Uyemura Intl Co.).

The baths in which the second coating takes place can include at least the metal ions, provided herein, and chemical additives. The solvent that is used in these baths can include water so that the bath is aqueous. Generally, deposition conditions such as the pH, deposition time, bath constituents, and deposition temperature can be chosen by those of ordinary skill in the art in order to achieve the desired thickness and composition of the second coating. For the various embodiments, the deposition conditions can be selected to provide the second coating with a predetermined thickness of greater than zero microns to 1 micron. Preferably, the predetermined thickness of the second coating is from 0.3 microns to 0.5 microns. For the various embodiments, the second coating can be continuous over the entire surface of the first coating.

In addition, the first coating and the second coating adhere to the surface of the base layer to achieve a 0 score on a bending test according to ASTM D522-93a test method B. This desirable property allows the metal based dental article of the present disclosure to be cut, bent, crimped, or otherwise manipulated by a practitioner without the coating (e.g., the first coating and the second coating) delaminating or cracking Therefore, use of the metal based dental article of the disclosure may allow a dental or orthodontic practitioner to precisely modify the fit or shape of the dental article without sacrificing performance or appearance.

The present disclosure also provides methods of forming the metal based dental article. The method includes providing the base layer formed from the metal substrate as discussed herein, where the base layer has a surface in the shape of the metal based dental article. If desired, the base layer of the metal substrate can be prepared prior to coating. For example, oil or other surface contaminants can be removed from the metal substrate by vapor degreasing, alkaline cleaning, acetone cleaning, or ultra-sonic cleaning, for example, as needed.

Surface oxides may also be removed and surface activation can be accomplished by acid treatment or abrasive blasting, for example, as are known and as described in more detail herein. Examples of suitable compositions for these purposed can include, but are not limited to, acids such as hydrochloric acid, nitric acids, hydrofluoric acids or ferric chlorides, among others. The acid treatment can also, in certain circumstances, help to impart surface roughness, as discussed herein, to the surface of the metal substrate. Specific examples of such acid treatment includes the use of an 50 weight percent (wt. %)/50 wt. % acid/water solution (e.g., 50 wt. %/50 wt. % hydrochloric acid aqueous solution) or commercially available cleaning solutions such as Asahi cleaner

C4000 from Uyemura Intl Co. or other solvent or alkali cleaners. After treating, the base layer of the metal substrate is rinsed with deionized water to remove the cleaning solution.

The first coating is electroplated on the surface of the base layer using one of the metals discussed above for the first coating. The first coating can also be formed using electroless plating when appropriate. As discussed above, the metal ions, solvent and plating additives present in the bath and the deposition conditions can be selected to form the first coating having a thickness of greater than zero microns to 25 microns. In addition to the thickness, the first coating also has a surface roughness (Rₐ) from 0.1 to 1.0 microns as measured according to ASTM B46.1-2009. Providing the first coating with the surface roughness (Rₐ) from 0.1 to 1.0 microns can be accomplished in one of a number of ways, as discussed herein. The second coating is then deposited by electroplating, as discussed herein, to a predetermined thickness on the first coating.

The combination of the first coating having the Rₐ from 0.1 to 1.0 microns and the second coating, each as described herein, provides the metal based dental article with a matte finish and a whiteness, L*, of at least 80 of the CIELAB color system (L*, a*, B*). So, for example, the second coating can provides the metal based dental article with the matte finish and a whiteness, L*, of 80 to 88 of the CIELAB color system (L*, a*, B*). Preferably, the second coating can provides the metal based dental article with the matte finish and a whiteness, L*, of 85 to 88 of the CIELAB color system (L*, a*, B*).

With respect to providing the first coating with the surface roughness as discussed herein, techniques for imparting the surface roughness can include, but are not limited to, chemical or mechanical techniques, where this treatment is applied to one or both of the base layer and/or the first coating of the metal based dental article. For example, prior to electroplating the first coating the base layer of the metal substrate can be sand, grit or bead blasted as known in the art by, for example, with 120-grit aluminum oxide sand, such as ROCATEC Pre, available from 3M ESPE. In some embodiments, the base layer may be subsequently treated with a silica-modified aluminum oxide (120-grit), such as ROCATEC Plus, also available from 3M ESPE. Alternatively, the base layer may be treated with ROCATEC Plus or other silica-modified aluminum oxide without prior treatment with ROCATEC Pre. Other techniques for sandblasting the base layer include those shown in U.S. Pat. No. 5,024,711 to Gasser et al. For electroplating purposes, a substrate can be basted with either glass beads or metal oxide grains as fine as 240-grit.

Alternatively, chemical techniques can be used to impart the desired surface roughness. Examples of suitable chemical techniques can include, but are not limited to, the acid treatment discussed herein, where the concentration of the acid solution, the time and temperature used during the acid treatment can create the desired surface roughness, as discussed herein (also see A. V. Pocius, C. J. Almer, R. D. Waid, T. H. Wilson, and B. E. Davidian, “Investigation of Variability in the Adhesive Bonding Characteristics of 301 Stainless Steel”, SAMPE Journal, November/December 1984, 11-16.). Alternatively, the chemical technique for imparting the surface roughness can include the use of a CL-Satin Nickel (Uyemura Intl Co.) electrolyte in forming the first coating.

Another approach to providing the first coating with the surface roughness can include fine particles dispersed in the deposited metal. The size, concentration, and composition of the particulate phase and of the total thickness of the metal layer can determine the appearance, adhesion, flexibility and wear resistance. For example, electroless nickel (EN) coating reinforced with diamond, silicon carbide, boron nitride, metal oxides, or PTFE particles (approximately 0.1 to 10 micron) can impart specific wear and lubricity properties for industrial and biomedical application (L. Ploof, “Composite Coatings,” Adv. Matls. And Proc., pp. 36-38, May 2008). Another method of providing the first coating with the surface roughness can include co-depositing multiple metals from the same plating bath. An example of such co-deposited first layer with a granular surface finish is Ni—Co, where different morphologies can be generated via electroplating conditions (A. Bai and C. Hu, “Effect of Electroplating variables on the composition and morphology of Ni—Co deposits plated through means of cyclic voltametry,” Electrochemica Acta 47 (2002) pp. 3447-3456). The use of chemical additive in electroplating is another avenue for generating surface textures of different roughness ranging from bright finish to a satin finish and/or a matte finish via modulation of crystallite size and/or intentional texturing.

As discussed herein, the metal substrate used in forming the metal based dental article of the present disclosure can have a base layer with a surface in the shape of a variety of dental restoratives (e.g., a pediatric crown). So, for example, providing the base layer can include selecting stainless steel as the metal substrate, where electroplating the first coating can include using nickel, as discussed herein, to form a satin nickel finish for the first coating, and electroplating the second coating can include using rhodium, as discussed herein, to form the matte finish and a whiteness, L*, of at least 80 of the CIELAB color system (L*, a*, B*) on the metal based dental article. The electroplating process discussed herein can be used to create the first coating with a thickness of greater than zero microns to 25 microns, and the second coating with a thickness of greater than zero microns to 1 micron.

Examples of commercially available base layers with surfaces in the shape of dental restoratives (e.g., a pediatric crown) can include, but are not limited to, preformed stainless steel crowns available from 3M Company (St. Paul, Minn.), Acero XT /(Dallas Tex.), Hu Friedy (Chicago, Ill.), mini and regular dental implants available from 3M Company (St Paul, Minn.), Zimmer Dental (Warsaw, Ind.), Straumann (Basel, Switzerland) and orthodontic brackets and archwires available from 3M Company (St. Paul, Minn.), American Orthodontics (Sheboygan, Wis.), Ormco (Orange, Calif.).

The metal based dental articles discussed herein can be implanted in a mouth of a patient. The shape and function of such metal based dental articles (e.g., a pediatric crown) is appreciated by one skilled in the art and the technique of implanting such metal based dental articles is known. As discussed herein, a significant advantage offered by the present disclosure is that the metal based dental articles to be implanted in the mouth of the patient have a coating that is thin, durable and esthetic so as to provide an inconspicuous and aesthetic appearance in situ. The coating is also flexible allowing for customization of the metal based dental article and thin enough to minimize loss of healthy hard tissue while implanting the metal based dental article.

The metal dental article (e.g., a pediatric crown) of the present disclosure can be provided in a kit along with at least one dental component selected from the group of a cement, an adhesive, an abrasive, an instrument, instructions for application of the metal dental article or a combination thereof. The kit may also include other components including, but not limited to, tools for the shaping and mounting of the metal based dental article in the mouth of the patient.

The methods discussed herein for forming the metal based dental article can also be used in treating the surfaces of other medical devices as well. For example, given a medical device that is at least partially formed from the metal substrate discussed herein, the metal substrate can be electroplated with the first coating and imparted to have the surface roughness (Rₐ) from 0.1 to 1.0 microns, where electroplating the second coating provides the metal substrate of the medical device with a matte finish and a whiteness, L*, of at least 80 of the CIELAB color system (L*, a*, B*). Examples of such medical devices include, but are not limited to, hand-held instruments (i.e., intraoral scanner wands), among other medical devices.

### EXAMPLES

Objects and advantages of this disclosure are further illustrated by the following examples, but the particular materials and amounts thereof recited in these examples, as well as other conditions and details, should not be construed to unduly limit this disclosure. Unless otherwise indicated, all parts and percentages are by weight and all molecular weights are weight average molecular weights.

Toothbrush Wear Test

Use a Toothbrush Wear Test (TWT) to determine the coating integrity of a coating, provided below, against prolonged toothbrush abrasion. Mount a square electroplated steel test coupon having a coating of the Example or Comparative Example provided herein (test sample) with double sized adhesive tape (Scotch Brand Tape, Core series 2-1300, St. Paul, Minn.) in a fixed specimen holder. Submerge the test sample in 5 milliliters (mL) of 50% by weight Crest™ toothpaste in water (toothpaste mixture). Subject the test sample to 40,000 to 60,000 strokes of a toothbrush head (a stroke is a 6 cm forward and back length), where the toothbrush head (47 Tuft Full Head (900-4.82), Acclean Gentle Care Toothbrushes, Henry Schein, Melville, N.Y.) is under a 250 gram (g) load. Measure each test sample for color (CIE Lab color space, L*a*b*) and 60 degree (°) gloss at the beginning and after each 5,000 strokes of the TWT. Rotate the test sample 90° after each set of 5,000 strokes, adding fresh toothpaste mixture before the next set of strokes. Replace brush heads every 15,000 strokes. Take five measurements per test sample at each test interval.

Bend Testing

Use the following modified ASTM D522-93a (test method B) test to characterize the crimping or bending characteristics of the Examples and Comparative Examples provided herein. Bend the Examples and Comparative Examples around a 1 millimeter (mm) diameter steel mandrel to an angle of 180° at a constant rate, such that the bend is accomplished in 1 to 2 seconds (s). Assess the extent of cracking of the coating on a scale from 0 to 4, with 0 representing no cracks and 4 representing complete delamination. A score of 0-1 (no cracks or a partial hairline crack at the edge) is a passing score.

Color Spectrum

Characterize the color of the surfaces of the Examples and Comparative Examples (as-received and/or after subjecting to the TWT) by the CIELAB color system (L*a*b*) using an X-Rite Spectrophotometer (X-Rite Color i7, Grand Rapids, Mich.). Use L* as a measure of whiteness (L* of the CIELAB color system (L*a*b*) is not the Whiteness Index (WI) of ASTM E313). The Commission Internationale De l′Eclairage defines the CIELAB color system (also known as CIE 1976 (L*, a*, b*) color space).

Gloss

Characterize the 60° gloss of the Examples and Comparative Examples with a glossmeter (Novo-Curve Glossmeter, Rhopoint Inst., East Sussex, England) for as-received and/or after the TWT. The instrument was calibrated with the manufacturer provides calibration standards (a black polymer foam surface and a glossy glass tile) before a set of TWT metal samples were measured.

Surface Roughness

Use a diamond stylus (0.7 micron dia.) profilometer [Dektak 6M, Veeco Instruments, Edina, Minn., USA] to measure the average roughness (Rₐ, microns) of the Examples and Comparative Examples as-received and/or after the TWT (take three measurements per Example or Comparative Example at each test interval or at the end of the TWT).

High Magnification Magnification Imaging with Elemental Analysis (EDS)

Analyze the surface morphology, dimensions of deposited layers and elemental analysis of the Examples and Comparative Examples using a scanning electron microscope (SEM) (Hitachi ™ 3000 Tabletop SEM with Bruker Quantax 70 EDS and an Energy Dispersive Spectroscopy). Perform surface, interface, and elemental analysis on the Examples and Comparative Examples as-received and after the TWT.

### Comparative Examples A through G (CE-A Through CE-G)

Comparative Example A (CE-A) is a 15.24 cm×2.54 cm×0.15 mm coupon of uncoated 304L stainless steel with 2B surface finish from AK Steel, West Chester, Ohio, USA. Use the uncoated 304L stainless steel as received.

Use the color spectrometer (X-Rite Color i7, Grand Rapids, Mich.) to quantify the appearance of Comparative Example A. As used herein, a natural tooth color is understood to have an average whiteness (L*) of approximately 72 (source “Color Distribution of Three Regions of Extracted Human Teeth,” O′Brian et al. Dent Mater, 1997 May 13(3): 179-85). As seen in Table 1, the L* value for Comparative Example A is 62.9.

Prepare Comparative Examples B through G (CE-B to CE-G) as follows. For each Comparative Example start with a 15.24 cm×2.54 cm×0.15 mm coupon of uncoated 304L stainless steel from AK Steel (West Chester, Ohio, USA). Each steel coupon was electroplated with Rh-Ru alloy for different times (Table 1) by Uyemura Intl Co., Southington, CT, USA per the following general procedure.

Remove oil and grease from the stainless steel coupons by soaking in Asahi Cleaner C-4000 for 1 to 2 minutes (min.) followed by 8 Volt anodic polarity for 30 seconds (s) at 120° F. to 140° F. Rinse the stainless steel coupons with deionized water and acid activate the stainless steel coupons in 50 wt. % hydrochloric acid for 1 to 2 min. After rinsing in deionized water, electroplate plate Gold Strike (Au/Co plating with Auruna-311, Uyemura Intl Co., Southington, CT, USA) at 2 to 6 amps per square foot (ASF) for 2 min on the stainless steel coupons. Rinse the electroplated stainless steel coupons in deionized water and deposit Rhoduna Alloy-1 (Rh/Ru at 4:1 ratio alloy from Uyemura Intl Co., Southington, CT, USA) at 3.5 Volt for a specified periods (0.5 min to 10 min). Rinse the electroplated stainless steel coupons in deionized water and dry them.

Table 1 summarizes the electroplating conditions of rhodium-ruthenium on the stainless steel coupons for each of Comparative Examples B through G. Table 1 also provides the color spectrum values (specular excluded) and 60° gloss values for Comparative Examples A through G. As seen in Table 1, with increasing plating time (or thickness) Comparative Examples B through G became glossier and less white. So, a rhodium-ruthenium coating alone on the stainless steel coupons is not sufficient to meet the color requirements (L*a*b*) for a natural tooth color with its average whiteness (L*) of approximately 72. In addition, the rhodium-ruthenium coating is glossier than the uncoated 304L stainless steel of Comparative Example A.

TABLE 1

| Ex- | | | Plating | CIE Lab | | | |
|---|---|---|---|---|---|---|---|
| am- | | Electroplated | Time, | Color Space | | | Gloss |
| ple | Substrate | Metal | min | L* | a* | b* | (60°) |
| CE-A | 304L | None | — | 62.9 | −0.62 | 1.15 | 271 |
| | Stainless | | | | | | |
| | Steel | | | | | | |
| CE-B | 304L | Rhodium- | 0.5 | 65.8 | −0.35 | −1.63 | 416 |
| | Stainless | Ruthenium | | | | | |
| | Steel | | | | | | |
| CE-C | 304L | Rhodium- | 1 | 61.09 | −0.33 | −1.49 | 464 |
| | Stainless | Ruthenium | | | | | |
| | Steel | | | | | | |
| CE-D | 304L | Rhodium- | 2 | 52.95 | −0.24 | −1.29 | 509 |
| | Stainless | Ruthenium | | | | | |
| | Steel | | | | | | |
| CE-E | 304L | Rhodium- | 3 | 44.11 | 0.06 | −0.44 | 523 |
| | Stainless | Ruthenium | | | | | |
| | Steel | | | | | | |
| CE-F | 304L | Rhodium- | 5 | 32.44 | 0.38 | 0.8 | 566 |
| | Stainless | Ruthenium | | | | | |
| | Steel | | | | | | |
| CE-G | 304L | Rhodium- | 10 | 29.61 | 0.41 | 0.88 | 626 |
| | Stainless | Ruthenium | | | | | |
| | Steel | | | | | | |

Table 2 shows the results of the TWT on Comparative Example B. The Moh's hardness of rhodium and ruthenium is about 6.0 and 6.5, respectively. As seen in Table 2, with prolonged toothbrush abrasion (the TWT) the rhodium-ruthenium coating did not exhibit any changes in the L* values. The rhodium-ruthenium coating is verified as being present on 304L stainless steel after 60,000 brush strokes as verified by EDS with no damage or delamination.

TABLE 2

| | TWT | CIE Lab Color Space | | | |
|---|---|---|---|---|---|
| Example | Strokes, # | L* | a* | b* | 60° Gloss |
| CE-B | 0 | 65.8 | −0.35 | −1.63 | 442.52 |
| | 5000 | 66.81 | −0.29 | −1.32 | 330.7 |
| | 10000 | 66.47 | −0.21 | −1.19 | 327.18 |
| | 20000 | 66.56 | −0.23 | −1.12 | 266.2 |
| | 40000 | 64.49 | −0.21 | −0.92 | 355.14 |
| | 45000 | 64 | −0.18 | −0.81 | 350.18 |

### Comparative Examples H and I

Prepare Comparative Examples H and I as follows. Comparative Example H was supplied by Uyemura Int Co, Southington, CT, with a 10 micron thick electroplated CL-Satin Nickel coating on a 15.24 cm×2.54 cm×0.15 mm coupon of uncoated 304L stainless steel from AK Steel (West Chester, Ohio, USA) per the following general procedure. Remove oil and grease from the stainless steel coupons by soaking in Asahi Cleaner C-4000 for 1 to 2 min. followed by additional treatment with 8 Volt anodic polarity for 30 s at 120° F. to 140° F. Rinse the stainless steel coupons with deionized water and acid activate the stainless steel coupons in 50 wt. % hydrochloric acid for 1 to 2 min. After rinsing in deionized water, electroplate Woods Nickel Strike at 40 ASF for 10 min. Rinse and electroplate CL-Satin Nickel with Additive-1 (Uyemura Int Co, Southington, CT) to 10 micron thick at 20 ASF for 10-15 min. Rinse the electroplated stainless steel coupons in deionized water and dry.

Comparative Example I was supplied by Prodigy Surface Tech, Santa Clara, Calif., with a 10 micron thick coating of satin nickel (via electroplating nickel sulfamate) without any plating additive on a 15.24 cm×2.54 cm×0.15 mm coupon of uncoated 304L stainless steel from AK Steel (West Chester, Ohio, USA).

Table 3 shows the color spectrum of Comparative Example I and Comparative Example J with whiteness, L*, better than the natural tooth.

TABLE 3

| | | 1st | | | | |
|---|---|---|---|---|---|---|
| | | Electroplated | | CIE Lab | | |
| | | Layer on | Plating | Color Space | | |
| Example | Substrate | Substrate | Additive | L* | a* | b* |
| CE-H | 304L | CL-Satin | A1 | 79.38 | 0.83 | 6.79 |
| | Stainless | Nickel | | | | |
| | Steel | | | | | |
| CE-I | 304L | Satin Nickel | None | 77.29 | 0.82 | 6.12 |
| | Stainless | | | | | |
| | Steel | | | | | |

Table 4 shows the results of the TWT on Comparative Example H and Comparative Example I. As can be seen from the data, with prolonged toothbrush abrasion the whiteness, L*, of both satin nickel coatings decrease with an accompanied increase in gloss. This suggests that the satin nickel coating is losing its matte finish with toothbrush abrasion. Both Comparative Example H and Comparative Example I coatings were present on the 304L stainless steel at the end of their respective toothbrush testing as verified by EDS with no damage or delamination.

TABLE 4

| | TWT | CIE Lab Color Space | | | |
|---|---|---|---|---|---|
| Example | Strokes, # | L* | a* | b* | 60° Gloss |
| CE-H | 0 | 79.38 | 0.83 | 6.79 | 20.52 |
| | 5000 | 73.9 | 1.07 | 8.09 | 28.46 |
| | 10000 | 76.67 | 0.8 | 6.31 | 56.2 |
| | 20000 | 72.82 | 0.73 | 5.94 | 96.56 |
| | 40000 | 63.67 | 0.64 | 5.31 | 174.12 |
| | 45000 | 59.47 | 0.67 | 5.32 | 202.6 |
| CE-I | 0 | 77.29 | 0.82 | 6.12 | 52.4 |
| | 5000 | 71.6 | 0.4 | 4.4 | 66.9 |
| | 10000 | 69.2 | 0.4 | 4.5 | 99.2 |
| | 20000 | 58.5 | 0.1 | 3.2 | 162.6 |
| | 30000 | 49.1 | 0.1 | 2.4 | 243.5 |
| | 40000 | 50.0 | −0.3 | 1.5 | 296.4 |
| | 50000 | 49.0 | −0.4 | 1.5 | 271.4 |

### Example 1 through 3

Prepare Examples 1 through Example 3, as follows. Example 1 was supplied by Uyemura Int Co, Southington, CT, with a first coating of 10 micron thick electroplated CL-Satin Nickel on a 15.24 cm×2.54 cm×0.15 mm coupon of uncoated 304L stainless steel from AK Steel (West Chester, Ohio, USA) followed by 0.5 micron rhodium-ruthenium second coating per the following general procedure.

Remove oil and grease from the stainless steel coupons by soaking in Asahi Cleaner C-4000 for 1 to 2 min. followed by additional treatment with 8 Volt anodic polarity for 30 s at 120° F. to 140° F. Rinse the stainless steel coupons with deionized water and acid activate the stainless steel coupons in 50 wt. % hydrochloric acid for 1 to 2 min. After rinsing in deionized water, electroplate Woods Nickel Strike at 40 ASF for 10 min. Rinse and electroplate CL-Satin Nickel with Additive-1 (Uyemura Int Co) to 10 micron thick at 20 ASF for 10-15 min. Rinse the electroplated stainless steel coupons in deionized water. Electroplate Rhoduna Alloy-1 (rhodium:ruthenium=4:1) at 3.5 volts to a thickness of 0.5 micron. Rinse in deionized water and dry.

Example 2 was supplied by Prodigy Surface Tech, Santa Clara, Calif. with a 10 micron electroplating nickel sulfamate (satin nickel finish) first coating on a 15.24 cm×2.54 cm×0.15 mm coupon of uncoated 304L stainless steel from AK Steel (West Chester, Ohio, USA), per Comparative Example J, followed by 0.5 micron electroplated rhodium second coating.

Example 3 was supplied by Professional Plating Inc., Anoka, Minn., on an acid etched 15.24 cm×2.54 cm×0.15 mm coupon of uncoated 304L stainless steel from AK Steel (West Chester, Ohio, USA) followed by electroplating Woods Nickel Strike, 3 micron thick first coating of nickel sulfamate (satin nickel finish), and a 0.25 micron thick second coating of rhodium-palladium.

Table 5 provides a summary of the electroplating conditions for generating Examples 1 through 3 of the present disclosure and their corresponding color spectrums. As can be seen from the data, Examples 1 through 3 have a whiteness, L*, higher than the average whiteness (L*) of a natural tooth.

TABLE 5

| | | | | 2nd | | | |
|---|---|---|---|---|---|---|---|
| | | | Ni | Coating | CIE Lab | | |
| | | 1st Coating on | Plating | over 1st | Color Space | | |
| Example | Substrate | Substrate | Additive | Coating | L* | a* | b* |
| 1 | 304L Stainless | CL-Satin Nickel | A1 | Rhodium- | 87.52 | 0.7 | 1.18 |
| | Steel | | | Ruthenium | | | |
| | | | | (Rhoduna | | | |
| | | | | Alloy-1) | | | |
| 2 | 304L Stainless | Satin Nickel | None | Rhodium | 84.14 | 1.00 | 1.94 |
| | Steel | | | | | | |
| 3 | 304L Stainless | Satin Nickel | None | Rhodium- | 83.59 | 0.99 | 1.88 |
| | Steel | | | Palladium | | | |

Table 6 shows the results of the TWT on Examples 1 through 3. As seen in Table 6, the TWT did not significantly change the color of Examples 1 through 3 after 40,000 to 50,000 toothbrush strokes. There was an increase in gloss for Examples 2 and 3 after 40,000 to 50,000 toothbrush strokes, but the gloss of Example 1 was only slightly increased after 40,000 to 50,000 toothbrush strokes. This suggests that coating a whiter and stronger metal such as Rh-Ru, Ru or Rh-Pd can help to improve the appearance of the surface as well as protect against surface wear. The coatings of Examples 1 through 3 were present on present on 304L stainless steel at the end of their respective TWT as verified by EDS with no damage or delamination.

TABLE 6

| | TWT | CIE Lab Color Space | | | |
|---|---|---|---|---|---|
| Example | Strokes, # | L* | a* | b* | 60° Gloss |
| 1 | 0 | 87.52 | 0.7 | 1.18 | 27.68 |
| | 5000 | 86.57 | 0.76 | 1.6 | 29.06 |
| | 10000 | 87.17 | 0.72 | 1.5 | 28.42 |
| | 20000 | 85.39 | 0.86 | 2.02 | 31.14 |
| | 40000 | 87.07 | 0.73 | 1.53 | 33.64 |
| | 45000 | 87.25 | 0.72 | 1.46 | 35.28 |
| 2 | 0 | 84.14 | 1.00 | 1.94 | 43.6 |
| | 5000 | 83.9 | 1.0 | 2.3 | 15.6 |
| | 10000 | 84.1 | 1.0 | 2.1 | 41.4 |
| | 20000 | 81.9 | 1.1 | 3.0 | 56.5 |
| | 30000 | 82.9 | 0.8 | 1.7 | 71.3 |
| | 40000 | 81.1 | 0.8 | 1.9 | 80.4 |
| | 50000 | 80.8 | 0.7 | 1.6 | 109.5 |
| 3 | 0 | 80.73 | 1.36 | 3.33 | 55.4 |
| | 5000 | 83.57 | 0.95 | 1.94 | 74.6 |
| | 10000 | 81.04 | 1.23 | 2.7 | 79.9 |
| | 20000 | 82.69 | 0.93 | 1.93 | 89.6 |
| | 41000 | 77.95 | 1.17 | 2.44 | 86.3 |

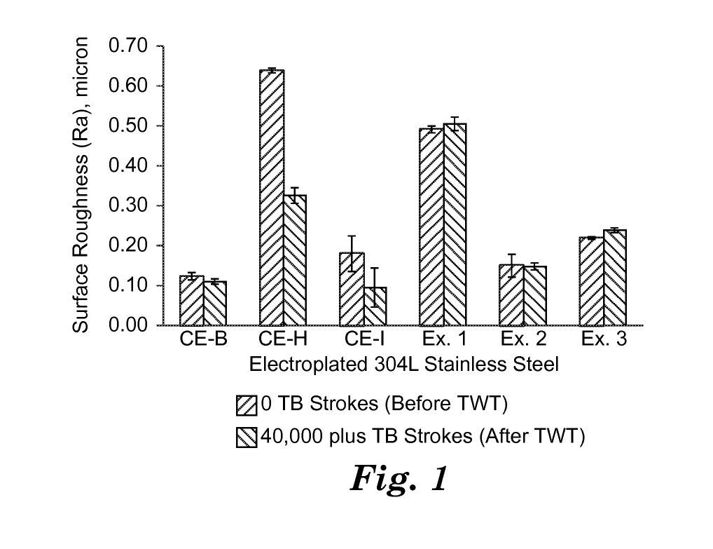

FIG. 1 shows the surface roughness of Examples 1 through 3 and of Comparative Examples B, H and I before and after the TWT. As seen in FIG. 1, Examples 1 through 3 and Comparative Example B show no reduction in surface roughness after 40,000 plus strokes of the TWT. However, Comparative Example B does not meet the whiteness, L*, requirement for a coating on 304L stainless steel when compared to whiter and robust coatings achieved in Examples 1 through 3. Comparative Examples H and I with satin nickel coatings showed a decrease (with Comparative Example H showing a significant decrease) in surface roughness after the same 40,000 plus strokes of the TWT. In addition, the satin nickel coating on the 304L stainless steel (Comparative Example H and I) exhibited a major loss of whiteness (L*) with an accompanying significant increase in gloss with prolonged toothbrush abrasion. However, Example 1 and Example 2 (satin-nickel with Rh/Ru or Rh for the second coating) exhibited a minimal loss of whiteness (L*), with the gloss of Example 1 being minimally changed with prolonged brushing.

From this data it is seen that Comparative Examples H and I (satin nickel plating) has improved esthetics (diffuse reflectance) approaching that of the average whiteness for a natural tooth, but does not have the wear resistance as evidenced by the increase in gloss at 60° with the TWT. Examples 1 through 3 demonstrate both a surface that is whiter than the average whiteness for a natural tooth and has improved toothbrush wear resistance over Comparative Example I. Each of Comparative Examples H and I and

Example 1 through Example 3 withstood the bending test described above, where each had a 0 score (no delamination or cracking) This is a direct result of robust adhesion to substrate and between layers.

### Example 4 and Comparative Examples J and K

Prepare Example 4 as follows. Repeat Comparative Example I and Example 2 with the following changes (supplied by Prodigy Surface Tech., Santa Clara, Calif.). Use prefabricated 0.15 mm thick 304L stainless steel crowns (L2 anterior crows from 3M ESPE, St. Paul, Minn.) in place of the 15.24 cm×2.54 cm×0.15 mm coupon of uncoated 304L stainless steel from AK Steel (West Chester, Ohio, USA). Table 7 shows the CIE Lab color spectrum (X-Rite Color i7, Grand Rapids, Mich.), specular excluded, of electroplated metal based dental article of Example 4 has an L* value of uncoated, satin-Ni coated, and Rh over satin-Ni coating were 65.29, 71.57, and 81.02, respectively.

TABLE 7

| | CIE Lab Color Space | | |
|---|---|---|---|
| Coated SSC (L2) | L* | a* | b* |
| CE J: SS Crown with no | 65.29 | 0.66 | 5.98 |
| coating | | | |
| CE K: SS Crown with Satin- | | | |
| Ni (no plating additive) | 71.57 | 1.33 | 7.42 |
| coating | | | |
| Ex 4: SS Crown with Satin- | 81.02 | 1.61 | 4.49 |
| Ni and Rh coatings | | | |

1. A metal based dental article, comprising:
a base layer formed from a metal substrate selected from the group consisting of a stainless steel, a titanium alloy or a nickel titanium alloy, the base layer having a surface in the shape of the metal based dental article;
a first coating on the surface of the base layer, the first coating formed from a metal selected from the group consisting of nickel, a nickel alloy, copper, a copper alloy, zinc, a zinc alloy, tin, a tin alloy, gold or a gold alloy, where
one of the first coating or the surface of the base layer has a surface
roughness (Ra) from 0.1 to 1.0 microns as measured according to ASTM B46.1-2009; and
a second coating on the first coating, the second coating formed from a metal selected from the group consisting of rhodium, a rhodium alloy, ruthenium, a ruthenium alloy, cobalt, a cobalt alloy, palladium, or a palladium alloy, where the second coating has a predetermined thickness that provides the metal based dental article with a matte finish and a whiteness, L*, of at least 80 of the CIELAB color system (L*, a*, B*).

2. The metal based dental article of claim 1, where the first coating is formed from nickel having a satin finish.

3. The metal based dental article of claim 1, where the first coating is formed from cobalt or a cobalt alloy, the first coating having a satin finish.

4. The metal based dental article of claim 1, where the first coating is formed from an alloy that includes copper, zinc and tin.

5. The metal based dental article of claim 2, where the first coating has a thickness from greater than zero microns to 25 microns.

6. The metal based dental article of claim 5, where the first coating has a thickness from 5 microns to 10 microns.

7. The metal based dental article of claim 1, where the second coating is formed from rhodium.

8. The metal based dental article of claim 7, where the second coating is formed from rhodium and ruthenium.

9. The metal based dental article of claim 7, where the second coating is formed from rhodium and palladium.

10. The metal based dental article of claim 1, where the predetermined thickness of the second coating is greater than zero microns to 1 micron.

11. The metal based dental article of claim 10, where the predetermined thickness of the second coating is from 0.3 microns to 0.5 microns.

12. The metal based dental article of claim 1, where the base layer is stainless steel selected from the group of a 304L stainless steel or a 316 medical grade stainless steel.

13. The metal based dental article of claim 1,where the first coating and the second coating adhere to the surface of the base layer to achieve a 0 score on a bending test according to ASTM D522-93a test method B.

14. The metal based dental article of claim 1, where the surface of the metal based dental article has the shape of a full crown, a partial crown, a pediatric crown, a bridge, a denture, an implant, an implant abutment, an implant healing cap, a post, a temporary restoration, an orthodontic bracket, an arch wire or a part of any one of those articles.

15. A method of forming a metal based dental article, comprising:
providing a base layer formed from a metal substrate selected from the group consisting of a stainless steel, a titanium alloy or a nickel titanium alloy, the base layer having a surface in the shape of the metal based dental article;
electroplating a first coating on the surface of the base layer using a metal selected from the group consisting of nickel, a nickel alloy, copper, a copper alloy, zinc, a zinc alloy, tin, a tin alloy, gold or a gold alloy;
providing the first coating with a surface roughness (Ra) from 0.1 to 1.0 microns as measured according to ASTM B46.1-2009; and
electroplating a second coating to a predetermined thickness on the first coating using a metal selected from the group consisting of rhodium, a rhodium alloy, ruthenium, a ruthenium alloy, cobalt, a cobalt alloy, palladium, or a palladium alloy, where the second coating provides the metal based dental article with a matte finish and a whiteness, L*, of at least 80 of the CIELAB color system (L*, a*, B*).

16. The method of claim 15, where providing the first coating with the surface roughness includes sandblasting the surface of the base layer to provide the surface roughness from 0.1 to 1.0 microns as measured according to ASTM B46.1-2009.

17. The method of claim 15, where providing the base layer includes selecting stainless steel as the metal substrate;
where electroplating the first coating includes using nickel to form a satin nickel finish for the first coating; and
where electroplating the second coating includes using rhodium to form the matte finish and a whiteness, L*, of at least 80 of the CIELAB color system (L*, a*, B*).

18. The method of claim 16, including electroplating the first coating to a thickness of greater than zero microns to 25 microns; and
electroplating the second coating to a thickness of greater than zero microns to 1 micron.

19. A method, comprising:
acquiring a metal based dental article of any one of claims 1 through Fl claim 1; and
implanting the metal based dental article in a mouth of a patient.

20. A kit, comprising:
a metal dental article of any one of claims 1 through Fl claim 1; and
at least one dental component selected from the group of a cement, an adhesive, an abrasive, an instrument, instructions for application of the metal dental article or a combination thereof.
